(19) United States
(12) Patent Application Publication
(10) Pub. No.: US 2025/0272797 A1
(43) Pub. Date: Aug. 28, 2025

(54) MEDICAL IMAGE PROCESSING APPARATUS, MEDICAL IMAGE PROCESSING METHOD, AND STORAGE MEDIUM

(71) Applicant: CANON MEDICAL SYSTEMS CORPORATION, Otawara-shi (JP)

(72) Inventor: Ryota OSUMI, Nasushiobara (JP)

(73) Assignee: CANON MEDICAL SYSTEMS CORPORATION, Otawara-shi (JP)

(21) Appl. No.: 19/063,439

(22) Filed: Feb. 26, 2025

(30) Foreign Application Priority Data
Feb. 28, 2024 (JP) 2024-028799

(51) Int. Cl. G06T 5/60; G06T 7/00

(52) U.S. Cl. CPC G06T 5/60; G06T 7/0014; G06T 2207/20048; G06T 2207/20081; G06T 2207/20084; G06T 2207/20092; G06T 2207/30004

(57) ABSTRACT

A medical image processing apparatus of an embodiment includes processing circuitry. The processing circuitry acquires an input image to be processed. The processing circuitry generates a reference image from the input image using a trained model. The processing circuitry adjusts the image quality of the input image. The processing circuitry compares the image-quality-adjusted image, which is the input image with the adjusted image quality, with the reference image and calculates an image quality difference between the images. The trained model is a machine learning model trained on the basis of a training data set including two unpaired images. The processing circuitry readjusts the image quality of the input image on the basis of the image quality difference.

### CROSS-REFERENCE TO RELATED APPLICATION

Priority is claimed on Japanese Patent Application No. 2024-028799, filed Feb. 28, 2024, the content of which is incorporated herein by reference.

### FIELD

Embodiments disclosed in this specification and drawings relate to a medical image processing apparatus, a medical image processing method, and a storage medium.

### BACKGROUND

One of the requirements for optimizing image quality in medical image diagnostic systems (also called medical image diagnostic apparatuses) is to cause an image generated by a system (hereinafter referred to as an input image) to come close to having the image quality of an image generated by another system (hereinafter referred to as a target image).

If an input image and a target image are in a paired relationship, parameters of a machine learning model can be optimized such that the image quality of the input image becomes close to the image quality of the target image on the basis of known reference-type indices such as most apparent distortion (MAD) and structural similarity (SSIM).

A “pair” mentioned here refers to a state in which an image in a source domain (i.e., an input image) and a corresponding image in a target domain (i.e., a target image) are paired with each other on a pixel-by-pixel basis.

However, in reality, it is a very special situation for an input image and a target image to be in a paired relationship. Therefore, it is difficult to use a paired input image and target image as a training data set to train a machine learning model.

Furthermore, in generating an image with reduced noise (an image with higher image quality) from an input image using a machine learning model, there are problems that a process through which the machine learning model performs calculations is not sufficiently explained and behaviors in unexpected situations are unclear. That is, image generation using a machine learning model may lack transparency and explainability.

On the other hand, instead of using a reference-type index, it is possible to optimize image quality using a known non-reference-type index such as a blind/referenceless image spatial quality evaluator (RISQUE), but its application to medical images has not been fully considered.

### BRIEF DESCRIPTION OF THE DRAWINGS

FIG. 1 is a diagram showing an example of a configuration of a medical image processing apparatus according to a first embodiment.

FIG. 2 is a flowchart showing a flow of a series of processing in processing circuitry of the medical image processing apparatus according to the first embodiment.

FIG. 3 is a diagram schematically showing the flow of a series of processing in the processing circuitry of the medical image processing apparatus according to the first embodiment.

FIG. 4 is a diagram illustrating a trained model according to the first embodiment.

FIG. 5 is a diagram showing an example of a display screen.

FIG. 6 is a diagram illustrating a trained model according to a second embodiment.

### DETAILED DESCRIPTION

Hereinafter, a medical image processing apparatus, a medical image processing method, and a storage medium of embodiments will be described with reference to the drawings.

The medical image processing apparatus of the embodiments includes processing circuitry. The processing circuitry acquires an input image to be processed.

The processing circuitry generates a reference image from the input image using a trained model. The processing circuitry adjusts the image quality of the input image. The processing circuitry compares the image-quality-adjusted image, which is the input image with the adjusted image quality, with the reference image, and calculates an image quality difference that is a difference between the image quality of the image-quality-adjusted image and the image quality of the reference image. The trained model is a machine learning model trained on the basis of a training data set including a training input image, which is an input image for training, and a training target image, which is a target image for training and is not paired with the training input image. The processing circuitry readjusts the image quality of the input image on the basis of the image quality difference. With this configuration, it is possible to generate medical images with high image quality using a machine learning model and further ensure transparency and explainability in the generation process.

### First Embodiment

### [Configuration of Medical Image Processing Apparatus]

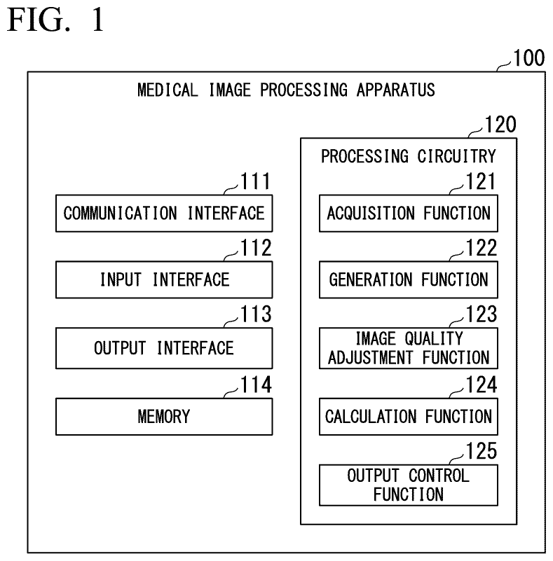

FIG. 1 is a diagram showing an example of a configuration of a medical image processing apparatus 100 according to a first embodiment. For example, the medical image processing apparatus 100 may be incorporated in a medical image diagnostic apparatus (also called a modality), or may be prepared separately from the medical image diagnostic apparatus. The medical image diagnostic apparatus may include, for example, an ultrasound diagnostic apparatus, an X-ray computed tomography (CT) apparatus, a magnetic resonance imaging (MRI) apparatus, an X-ray apparatus, and the like.

The medical image processing apparatus 100 may be a single apparatus or may be a system in which a plurality of apparatuses connected via a communication network NW operate in cooperation with each other.

The communication network NW may refer to a general information and communication network that utilizes electric communication technology. For example, the communication network NW includes telephone communication line networks, optical fiber communication networks, cable communication networks, and satellite communication networks in addition to wireless/wired local area networks (LANs) such as a hospital backbone LAN and the Internet.

That is, the medical image processing apparatus 100 may be realized by a plurality of computers (processors) included in a distributed computing system or a cloud computing system.

As shown in the figure, for example, the medical image processing apparatus 100 includes a communication interface 111, an input interface 112, an output interface 113, a memory 114, and processing circuitry 120.

The communication interface 111 communicates with an external device via the communication network NW. The communication interface 111 includes, for example, a network interface card (NIC), an antenna for wireless communication, and the like.

The input interface 112 receives various input operations from an operator, converts the received input operations into electrical signals, and outputs the electrical signals to the processing circuitry 120.

For example, the input interface 112 includes a mouse, a keyboard, a trackball, a switch, a button, a joystick, a touch panel, and the like. The input interface 112 may be, for example, a user interface that receives audio input, such as a microphone. If the input interface 112 is a touch panel, the input interface 112 may also have the display function of a display 113a included in the output interface 113 which will be described later.

In this specification, the input interface 112 is not limited to one equipped with physical operating parts such as a mouse and a keyboard. Examples of the input interface 112 also include electrical signal processing circuitry that receives an electrical signal corresponding to an input operation from external input equipment provided separately from the apparatus and outputs the electrical signal to a control circuit, for example.

The output interface 113 includes, for example, a display 113a, a speaker 113b, and the like. The display 113a displays various types of information.

For example, the display 113a displays images generated by the processing circuitry 120, a graphical user interface (GUI) for receiving various input operations from the operator, and the like. For example, the display 113a is a liquid crystal display (LCD), a cathode ray tube (CRT) display, an organic electroluminescence (EL) display, or the like. The speaker 113b outputs information input from the processing circuitry 120 as sound.

The memory 114 is realized, for example, by a semiconductor memory element such as a random access memory (RAM) or a flash memory, a hard disk, or an optical disc. Such non-transient storage media may be realized by other storage devices connected via the communication network NW, such as a network attached storage (NAS) and an external storage server device.

The memory 114 may also include non-transient storage media such as a read only memory (ROM) and a register. The memory 114 stores programs executed by a hardware processor of the processing circuitry 120, various arithmetic results from the processing circuitry 120, model definition data, and the like.

The model definition data is information (program or algorithm) that defines a trained model MDL which will be described later.

The processing circuitry 120 includes, for example, an acquisition function 121, a generation function 122, an image quality adjustment function 123, a calculation function 124, and an output control function 125. The acquisition function 121 is an example of an “acquisition unit,” the generation function 122 is an example of a “generation unit,” the image quality adjustment function 123 is an example of an “image quality adjustment unit,” and the calculation function 124 is an example of a “calculation unit.”

The processing circuitry 120 realizes these functions by, for example, a hardware processor (computer) executing a program stored in the memory 114 (storage circuit).

The hardware processor in the processing circuitry 120 refers to circuitry such as a central processing unit (CPU), a graphics processing unit (GPU), an application specific integrated circuit (ASIC), or a programmable logic device (e.g., a simple programmable logic device (SPLD) or a complex programmable logic device (CPLD), or a field programmable gate array (FPGA)).

Instead of storing the program in the memory 114, the program may be directly built into the circuit of the hardware processor. In this case, the hardware processor realizes functions by reading and executing the program built into the circuit. The aforementioned program may be stored in memory 114 in advance, or may be stored in a non-transient storage medium such as a DVD or CD-ROM and installed into the memory 114 from the non-transient storage medium by setting the non-transient storage medium in a drive device (not shown) of the medical image processing apparatus 100.

The hardware processor is not limited to being configured as a single circuit, but may be configured as one hardware processor by combining a plurality of independent circuits to realize each function. Further, a plurality of components may be integrated into one hardware processor to realize each function.

### [Processing Flow of Medical Image Processing Apparatus]

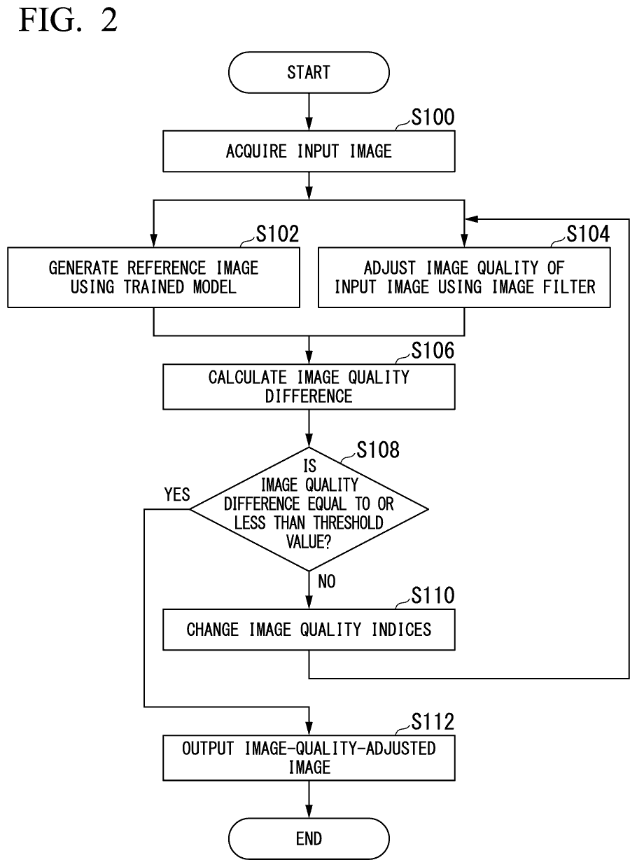

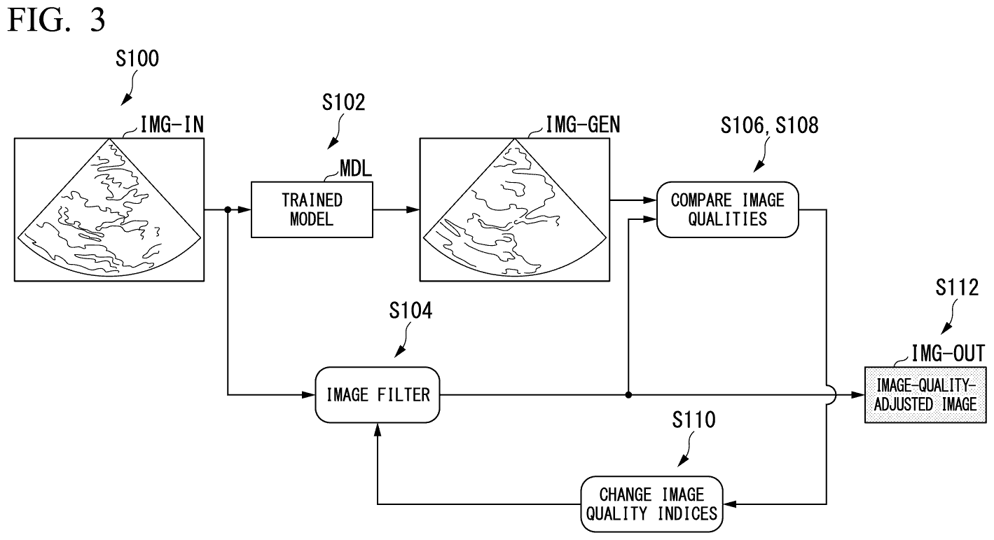

Hereinafter, a series of processing performed by the processing circuitry 120 of the medical image processing apparatus 100 will be described using FIG. 2 and FIG. 3. FIG. 2 is a flowchart showing a flow of a series of processing of the processing circuitry 120 of the medical image processing apparatus 100 according to the first embodiment. FIG. 3 is a diagram schematically showing the flow of a series of processing of the processing circuitry 120 of the medical image processing apparatus 100 according to the first embodiment.

First, the acquisition function 121 acquires an input image IMG-IN (step S100). The input image IMG-IN is a medical image obtained by scanning a subject with a medical image diagnostic apparatus (modality). The subject is typically a human being, but is not limited thereto and may be other animals such as a dog and a cat, or may be a plant.

For example, if the medical image diagnostic apparatus is an ultrasound diagnostic apparatus, the input image IMG-IN is an ultrasound image. Similarly, the input image IMG-IN is a CT image if the medical image diagnostic apparatus is an X-ray CT apparatus, the input image IMG-IN is an MR image if the medical image diagnostic apparatus is an MRI apparatus, and the input image IMG-IN is an X-ray image if the medical image diagnostic apparatus is an X-ray apparatus.

Next, the generation function 122 reads model definition data from the memory 114 and generates a reference image IMG-GEN from the input image IMG-IN using a trained model MDL defined by the model definition data (step S102).

The reference image IMG-GEN is a medical image that is output by the trained model MDL in response to the input image IMG-IN being input to the trained model MDL, and is a medical image that is referenced during image quality comparison processing which will be described later.

The trained model MDL may be realized, for example, by Cycle-Consistent Adversarial Networks (CycleGAN), a model derived from CycleGAN (for example, Cycle-MedGAN), or the like. The trained model MDL may be implemented using other machine learning models such as a support vector machine, a decision tree, a random forest, or a logistic regression, instead of a neural network such as CycleGAN. In the following, an example in which the trained model MDL is implemented by CycleGAN will be described.

When the trained model MDL is implemented by a neural network such as CycleGAN, the model definition data includes, for example, coupling information regarding how units included in each layer of an input layer, one or more hidden layers (intermediate layers), and an output layer that constitute the neural network are coupled to each other, weight information regarding a coupling coefficient assigned to data input and output between coupled units

The coupling information includes, for example, the number of units included in each layer, information specifying the type of a unit that is a coupling destination of each unit, an activation function that realizes each unit, gates provided between units in the hidden layer, and the like.

The activation function that realizes units may be, for example, a rectified linear unit (ReLU) function, an exponential linear units (ELU) function, a clipping function, a sigmoid function, a step function, a hyperbolic tangent function, an identity function, or the like. The gates selectively pass or weight data transmitted between units, for example, depending on a value (e.g., 1 or 0) returned by the activation function.

The coupling coefficient includes, for example, a weight assigned to output data when data is output from a unit in a certain layer to a unit in a deeper layer in a hidden layer of the neural network. Further, the coupling coefficient may also include a bias component specific to each layer, and the like.

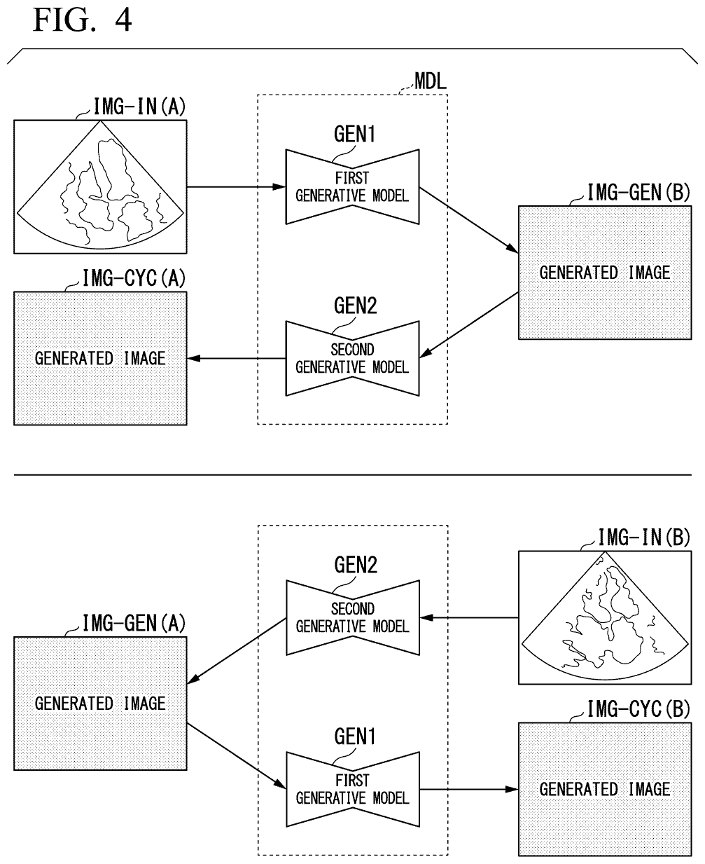

FIG. 4 is a diagram illustrating the trained model MDL according to the first embodiment. For example, when the trained model MDL is implemented by CycleGAN, the trained model MDL includes a first generative model GEN1 and a second generative model GEN2 as illustrated in the figure. The first generative model GEN1 and the second generative model GEN2 are implemented by a combination of an encoder and a decoder.

The first generative model GEN1 and the second generative model GEN2 are trained on the basis of a training data set composed of two unpaired images.

As described above, a pair refers to a state in which an image in a source domain (i.e., an input image) and a corresponding image in a target domain (i.e., a target image) are paired with each other on a pixel-by-pixel basis.

For example, a medical image before specific image processing is performed and a medical image after specific image processing is performed are paired with each other. If the medical image is an ultrasound image, an ultrasound image generated from ultrasound signal data before beam forming (BF) processing according to a certain BF method and an ultrasound image generated from the same ultrasound signal data before BF processing according to another BF method are paired with each other. Similarly, an ultrasound B-mode and a color Doppler, contrast, elastography, or attenuation image of the same time phase are paired with each other. If the medical image is an MR image, a T1-emphasized image and a T2-emphasized image of the same original MR scan data are also paired with each other.

In the present embodiment, such paired images are not used as a training data set, but rather images in an unpaired relationship are used as a training data set. That is, an image in a source domain (i.e., an input image) and an image in a target domain (i.e., a target image) that is not paired on a pixel-by-pixel basis are used as a training data set.

For example, the first generative model GEN1 and the second generative model GEN2 are trained using a training data set composed of a training input image IMG-IN(A) and a training target image IMG-IN(B). The training input image IMG-IN(A) is an input image of a certain source domain A prepared for training. The training target image IMG-IN(B) is a target image of a certain target domain B prepared for training and is an unpaired with the training input image IMG-IN(A).

### [Forward Image Transformation Process]

The training input image IMG-IN(A) is input to the first generative model GEN1. In response, the first generative model GEN1 transforms the training input image IMG-IN(A) into an image close to the target domain B and outputs the same. Hereinafter, an image transformed from the training input image IMG-IN(A) by the first generative model GEN1 and close to the target domain B will be referred to as the generated image IMG-GEN(B).

The generated image IMG-GEN(B) output by the first generative model GEN1 is input to the second generative model GEN2. In response, the second generative model GEN2 transforms the generated image IMG-GEN(B) into an image close to the source domain A and outputs the same. Hereinafter, an image transformed by the first generative model GEN1 into an image close to the target domain B and then further transformed by the second generative model GEN2 into an image close to the original source domain A will be referred to as a generated image IMG-CYC(A).

### [Reverse Image Transformation Process]

Meanwhile, the training target image IMG-IN(B) is also input to the second generative model GEN2. In response, the second generative model GEN2 transforms the training target image IMG-IN(B) into an image close to the source domain A and outputs the same. Hereinafter, an image transformed from the training target image IMG-IN(B) by the second generative model GEN2 and close to the source domain A is referred to as a generated image IMG-GEN(A).

The generated image IMG-GEN(A) output by the second generative model GEN2 is also input to the first generative model GEN1. In response, the first generative model GEN1 transforms the generated image IMG-GEN(A) into an image close to the target domain B and outputs the same. Hereinafter, an image transformed by the second generative model GEN2 into an image close to the source domain A, and then further transformed by the first generative model GEN1 into an image close to the original target domain A will be referred to as a generated image IMG-CYC(B).

Parameters (weighting coefficients and bias components) of the first generative model GEN1 and the second generative model GEN2 described above are adjusted on the basis of a loss in the forward image transformation process and a loss in the reverse image transformation process. As described above, the forward image transformation process is cyclic processing of transforming the training input image IMG-IN(A) into a generated image IMG-GEN(B), which is an image close to the target domain B, and further transforming the generated image IMG-GEN(B) into a generated image IMG-CYC(A), which is an image close to the source domain A. The reverse image transformation process is cyclic processing of transforming the training target image IMG-IN(B) into a generated image IMG-GEN(A), which is an image close to the source domain A, and further transforming the generated image IMG-GEN(A) into a generated image IMG-CYC(B), which is an image close to the target domain B.

For example, the parameters (weighting coefficients and bias components) of the first generative model GEN1 and the second generative model GEN2 are adjusted such that the total loss, which is the sum of the loss in the forward image transformation process and the loss in the reverse image transformation process, is minimized. Such total loss is a so-called cycle consistency loss and adversarial loss.

By training the first generative model GEN1 and the second generative model GEN2 using a training data set composed of two such unpaired images, it is possible to generate a reference image IMG-GEN that is not paired with the input image IMG-IN on a pixel-by-pixel basis in the process of S102. More specifically, the generation function 122 inputs the input image IMG-IN to the first generative model GEN1 between the trained first generative model GEN1 and second generative model GEN2, thereby generating the reference image IMG-GEN that is in a different domain from the source domain of the input image IMG-IN and is unpaired.

Returning to the description of FIG. 2 and FIG. 3, the image quality adjustment function 123 adjusts the image quality of the input image IMG-IN using an image filter (step S104). Hereinafter, the input image IMG-IN whose image quality has been adjusted using the image filter will be referred to as an image-quality-adjusted image IMG-OUT.

The image filter is a filter for smoothing the input image IMG-IN to reduce noise and for emphasizing a feature such as edges. The feature to be emphasized is, for example, edges of a specific biological tissue such as a blood vessel.

Next, the calculation function 124 compares the reference image IMG-GEN generated using the trained model MDL with the image-quality-adjusted image IMG-OUT and calculates the difference (hereinafter referred to as an image quality difference) between the image quality of the reference image IMG-GEN and the image quality of the image-quality-adjusted image IMG-OUT (step S106).

Next, the calculation function 124 judges whether the image quality difference is equal to or less than a threshold value (step S108). The threshold value may be, for example, a value at which the image quality difference is considered to be sufficiently small.

Upon determining that the image quality difference exceeds the threshold value, the image quality adjustment function 123 changes all or some of a plurality of image quality indices referenced when adjusting the image quality of the input image IMG-IN such that the image quality difference decreases (step S110). Accordingly, the image quality of the input image IMG-IN is readjusted.

The image quality indices include a degree of smoothing of the image filter described above, a degree of feature emphasis, and the like. The image quality indices may be interpreted as parameters of the image filter.

When the image quality of the input image IMG-IN is readjusted by changing the image quality indices, the image quality difference between the reference image IMG-GEN and the image-quality-adjusted image IMG-OUT is recalculated. In this manner, adjustment of the image quality of the input image IMG-IN is repeated until the image quality difference becomes equal to or less than the threshold value.

On the other hand, upon determining that the image quality difference is equal to or less than the threshold value, the output control function 125 outputs the image-quality-adjusted image IMG-OUT that has been compared with the reference image IMG-GEN (step S112).

For example, the output control function 125 may cause the display 113a to display the image-quality-adjusted image IMG-OUT. Further, the output control function 125 may transmit the image-quality-adjusted image IMG-OUT to an external device (for example, a computer used by medical personnel such as doctors and engineers) via the communication interface 111. The output control function 125 may also output the reference image IMG-GEN in addition to the image-quality-adjusted image IMG-OUT. This ends processing of this flowchart.

According to the first embodiment described above, the processing circuitry 120 of the medical image processing apparatus 100 acquires an input image IMG-IN to be processed. The processing circuitry 120 generates a reference image IMG-GEN from the input image IMG-IN using the trained model MDL.

The trained model MDL is a machine learning model (e.g., CycleGAN) trained in advance using a training data set composed of a training input image IMG-IN(A) and a training target image IMG-IN(B). The training input image IMG-IN(A) is an input image of a certain source domain A prepared for training. The training target image IMG-IN(B) is a target image of a certain target domain B prepared for training and is unpaired with the training input image IMG-IN(A).

The processing circuitry 120 adjusts the image quality of the input image IMG-IN, compares the image-quality-adjusted image IMG-OUT, which is the input image IMG-IN with the adjusted image quality, with the reference image IMG-GEN generated using the trained model MDL, and calculates the image quality difference between the images. The processing circuitry 120 readjusts the image quality of the input image IMG-IN on the basis of the image quality difference.

In this manner, by adjusting the parameters (image quality indices) of the image filter such that the image quality of the image-quality-adjusted image IMG-OUT generated through the image filter approaches the image quality of the reference image IMG-GEN generated by a machine learning model such as CycleGAN, a medical image with high image quality can be generated. Furthermore, instead of finally outputting a reference image IMG-GEN generated by a machine learning model whose process is a black box, the image-quality-adjusted image IMG-OUT generated through a conventionally known image filter is output, and thus medical personnel and patients can be given a convincing explanation on the basis of conventional knowledge. That is, transparency and explainability can be ensured.

### Modified Example of First Embodiment

Hereinafter, a modified example of the first embodiment will be described. Although the image quality adjustment function 123 changes the image quality indices (parameters of the image filter) such that the image quality difference decreases when it is determined that the image quality difference exceeds the threshold value in the above description of the first embodiment, the present disclosure is not limited thereto. For example, the image quality adjustment function 123 may change scan conditions of the medical image diagnostic apparatus such that the image quality difference decreases.

When the medical image diagnostic apparatus is an ultrasound diagnostic apparatus, the scan conditions include, for example, on or off of a zoom function, a frame rate value, an image quality level, a dynamic range value, and a brightness value. The scan conditions may further include the type of an ultrasound probe used for scanning, presence or absence of a stress echo test, and the like.

In addition, when the medical image diagnostic apparatus is an X-ray CT apparatus, the scan conditions include, for example, a scan area, a scan method, conditions for collecting detection data or projection data, conditions for reconstructing a CT image, and the like.

In this manner, the image quality of the input image IMG-IN may be adjusted after acquisition using an image filter, or the image quality of the input image IMG-IN may be adjusted in advance by changing scan conditions before or during acquisition of the input image IMG-IN.

In the above description of the first embodiment, the image quality of the input image IMG-IN is automatically adjusted until the image quality difference is equal to or less than the threshold value, but the present disclosure is not limited thereto. For example, the image quality adjustment function 123 may adjust the image quality of the input image IMG-IN on the basis of image quality indices preset by a user such as a doctor or an engineer. That is, the image quality of the input image IMG-IN may be manually adjusted according to a user request.

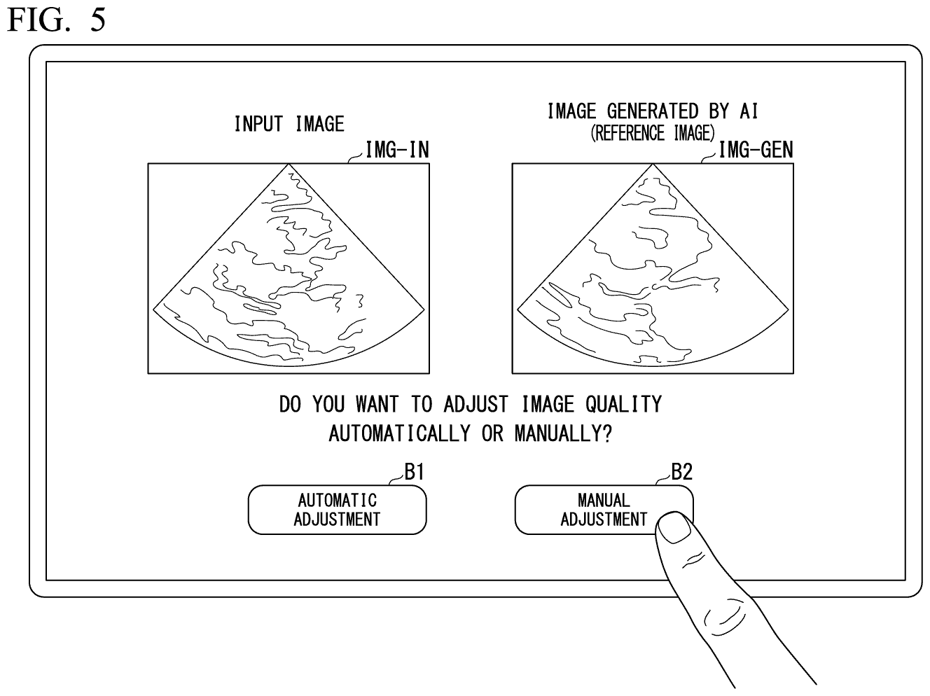

FIG. 5 is a diagram showing an example of a screen of the display 113a. As illustrated, the display 113a may display a graphical user interface (GUI) for allowing a user to select whether to automatically or manually adjust the image quality of the input image IMG-IN. For example, the current input image IMG-IN, which is a subject of calculation of the image quality difference, and the reference image IMG-GEN generated by the machine learning model may be displayed side by side on the GUI. For example, when the user operates a button B1 (automatic adjustment button) on the GUI, the image quality difference between these images is calculated, and when the image quality difference exceeds the threshold value, the image quality indices (parameters of the image filter) are automatically changed and the image quality of the input image IMG-IN is automatically readjusted. On the other hand, when the user operates a button B2 (manual adjustment button) on the GUI, the image quality of the input image IMG-IN is readjusted on the basis of image quality indices set by the user. In this manner, a user operation may be involved in adjustment of the image quality of the input image IMG-IN. That is, the image quality of the input image IMG-IN may be interactively adjusted.

### Second Embodiment

Hereinafter, a second embodiment will be described. In the above-described first embodiment, the trained model MDL is described as being implemented by CycleGAN. In contrast, the second embodiment differs from the first embodiment in that the trained model MDL is implemented by a diffusion model. Hereinafter, the differences from the first embodiment will be mainly described, and points in common with the first embodiment will not be described. In the description of the second embodiment, the same parts as those in the first embodiment will be described with the same reference numerals.

The generation function 122 according to the second embodiment generates a reference image IMG-GEN from the input image IMG-IN using a diffusion model. At this time, the generation function 122 controls the features of the reference image IMG-GEN on the basis of the features of the input image IMG-IN (for example, edges, segmentation, and the like).

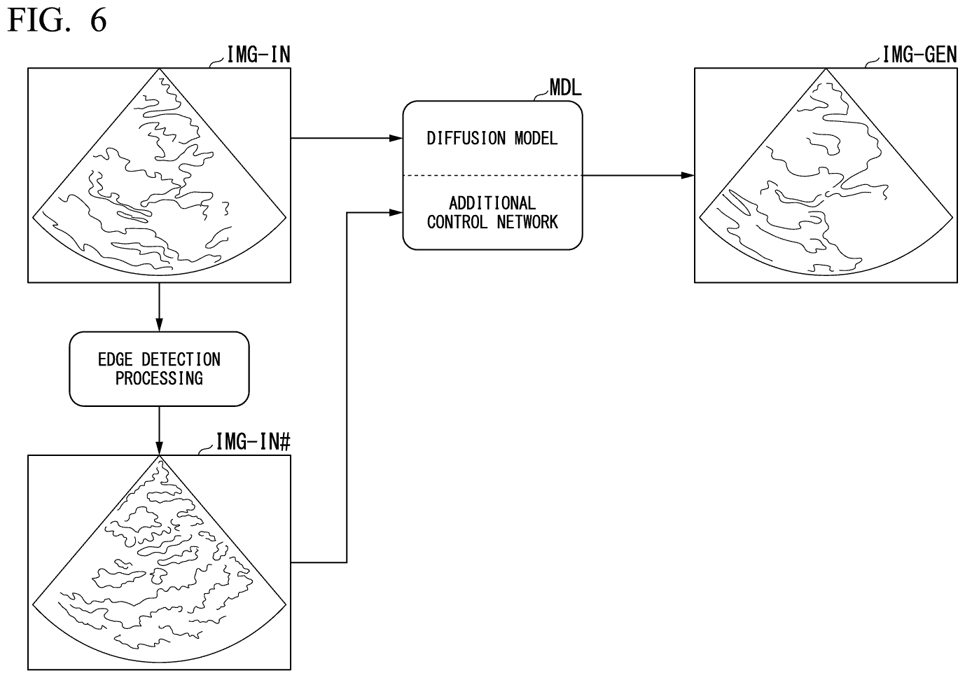

FIG. 6 is a diagram illustrating a trained model MDL according to the second embodiment. The trained model MDL according to the second embodiment is implemented by a diffusion model. The diffusion model is used for image generation, similar to CycleGAN.

In particular, the diffusion model may be a latent diffusion model that diffuses latent variables transformed from the input image IMG-IN by an arbitrary encoder in a latent space.

The diffusion model may be provided with a conditional mechanism that incorporates feature vectors encoded by other encoders by cross attention. In the present embodiment, a control net (also called a Stable Diffusion control network) is applied as the conditional mechanism. The control net is a model that applies a Stable Diffusion, which is a type of diffusion model.

First, the image quality adjustment function 123 according to the second embodiment performs edge detection processing (e.g., Canny Filter or the like) on the input image IMG-IN to generate an input image IMG-IN with enhanced edges.

Hereinafter, the input image IMG-IN with enhanced edges will be referred to as an edge-enhanced image IMG-IN #.

Next, the image quality adjustment function 123 according to the second embodiment inputs the input image IMG-IN to the diffusion model, which is the trained model MDL, and also inputs the edge-enhanced image IMG-IN # to a control net provided as a conditional mechanism for the diffusion model. This causes the diffusion model to generate a reference image IMG-GEN with edges controlled according to the edge-enhanced image IMG-IN # input to the control net. On the reference image IMG-GEN generated in this manner, biological tissues and the like are drawn along the edges of the edge-enhanced image IMG-IN #. As a result, it is possible to prevent the trained model MDL from generating a medical image (reference image IMG-GEN) that are unnatural to the human eye.

According to the second embodiment described above, even when a diffusion model is applied as a trained model MDL, it is possible to generate medical images with high image quality as in the first embodiment, and furthermore, it is possible to ensure transparency and explainability in the generation process.

Furthermore, according to the second embodiment, when a control net is applied to the conditional mechanism of the diffusion model, it is possible to curb generation of a medical image (reference image IMG-GEN) that are unnatural to the human eye.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical image processing apparatus comprising processing circuitry configured to:
acquire an input image to be processed;
generate a reference image from the input image using a trained model;
adjust an image quality of the input image;
compare an image-quality-adjusted image, which is the input image with the adjusted image quality, with the reference image; and
calculate an image quality difference, which is a difference between the image quality of the image-quality-adjusted image and the image quality of the reference image,
wherein the trained model is a machine learning model trained on the basis of a training dataset including a training input image, which is an input image for training, and a training target image, which is a target image for training and is not paired with the training input image, and the processing circuitry readjusts the image quality of the input image on the basis of the image quality difference.

2. The medical image processing apparatus according to claim 1, wherein the processing circuitry adjusts the image quality of the input image by changing all or some of a plurality of image quality indices referenced when adjusting the image quality of the input image such that the image quality difference decreases.

3. The medical image processing apparatus according to claim 1, wherein the input image is acquired by a medical image diagnostic apparatus scanning a subject,
wherein the processing circuitry adjusts the image quality of the input image by changing scanning conditions of the medical image diagnostic apparatus such that the image quality difference decreases.

4. The medical image processing apparatus according to claim 1, wherein the processing circuitry further adjusts the image quality of the image-quality-adjusted image in response to a user request.

5. The medical image processing apparatus according to claim 1, wherein training of the trained model includes:
a forward transformation process for transforming the training input image into the training target image;
a reverse transformation process for transforming the training target image into the training input image; and
an adjustment process for adjusting parameters of the machine learning model on the basis of a forward loss and a reverse loss.

6. The medical image processing apparatus according to claim 5, wherein the trained model is CycleGAN or a model derived from CycleGAN.

7. The medical image processing apparatus according to claim 1, wherein the processing circuitry controls features of the reference image on the basis of features of the input image when generating the reference image from the input image using the trained model.

8. The medical image processing apparatus according to claim 7, wherein the trained model is a diffusion model including a control net as a conditional mechanism,
wherein the diffusion model generates the reference image with controlled features according to the control net.

9. A medical image processing method using a computer, comprising:
acquiring an input image to be processed;
generating a reference image from the input image using a trained model;
adjusting the image quality of the input image; and
comparing an image-quality-adjusted image, which is the input image with the adjusted image quality, with the reference image, and calculating an image quality difference, which is a difference between the image quality of the image-quality-adjusted image and the image quality of the reference image,
wherein the trained model is a machine learning model trained on the basis of a training dataset including a training input image, which is an input image for training, and a training target image, which is a target image for training and is not paired with the training input image,
the medical image processing method further comprising readjusting the image quality of the input image on the basis of the image quality difference.

10. A computer-readable non-transient storage medium storing a program for causing a computer to execute:
acquiring an input image to be processed;
generating a reference image from the input image using a trained model;
adjusting the image quality of the input image; and
comparing an image-quality-adjusted image, which is the input image with the adjusted image quality, with the reference image, and calculating an image quality difference, which is a difference between the image quality of the image-quality-adjusted image and the image quality of the reference image,
wherein the trained model is a machine learning model trained on the basis of a training dataset including a training input image, which is an input image for training, and a training target image, which is a target image for training and is not paired with the training input image,
the computer further executing readjusting the image quality of the input image on the basis of the image quality difference.
